(19) United States
(12) Patent Application Publication
(10) Pub. No.: US 2016/0213429 A1
(43) Pub. Date: Jul. 28, 2016

(54) SYSTEM AND METHOD FOR SURGICAL PLANNING

(71) Applicant: KERA HARVEST INCORPORATION, CHIAYI (TW)

(72) Inventor: I-KU KUO, Chiayi (TW)

(21) Appl. No.: 14/603,668

(22) Filed: Jan. 23, 2015

(51) Int. Cl. A61B 19/00

(52) U.S. Cl. CPC A61B 19/5225; A61B 19/50; A61B 19/56; A61B 2019/524; A61B 2019/5276; A61B 2019/5231; A61B 2019/5289; A61B 2019/562

(57) ABSTRACT

The present invention relates to a system and method for surgical planning. Wherein, the system comprises a computer, an image capturing module and a warning module. The computer is connected to the image capturing module and the warning module, and generates a surgical planning information via a surgical planning program based on a pre-surgery image captured by the image capturing module. The image capturing module captures a surgery operation image during a surgery and a post-surgery image while the surgery is completed. The warning module generates a warning signal to a surgery operator according to the differences between the surgery operation image and the surgical planning information. The surgical planning program is updated based on the differences between the post-surgery image and the surgical planning information.

### BACKGROUND OF THE INVENTION

1. Field of the Invention

The present invention relates to a system and method for surgical planning, particularly to a system and method for surgical planning with effectiveness of warning assistance and system update.

2. Description of the Prior Art

As the development of the science, the knowledge and the technology are also improved accordingly and go in a direction of specialization. The complicated latter-day surgery is almost impossible to be completed by one or few people like that in the past.

Hence, several related systems and methods of surgical/medical assistance are proposed in recent years, and an assistance system integrating the software and hardware resources that formed by the professional medical knowledge and the related hardware equipment is expected. The surgical plan for evaluating, estimating or planning before the medical treatment or surgery can help to achieve the related medical treatment/surgery with less manpower or assist the surgery operators/practitioners/doctors who carry out the treatment to accomplish higher quality works. However, without any related update or feedback function, the conventional assistance systems or methods cannot be instantly or efficiently updated according to the previous actual experience which is different from the surgical plan. The later surgical plan may be unsuitable, and the surgery operator may bring out harmful treatment, or even serious result, when taking the unsuitable surgical plan.

### SUMMARY OF THE INVENTION

For improving the above-mentioned defects of the conventional technology, the present invention is directed to provide a system and method for surgical planning which is capable of providing an assistant warning signal through the warning module and updating the system through the related updating mechanisms.

In one broad aspect, there is provided a surgical planning system comprising: a computer having an accessible non-transitory memory and a surgical planning program, the surgical planning program is stored in the accessible non-transitory memory; an image capturing module directly or indirectly connected to the computer, and comprising at least one of a camera and a video camera to capture a pre-surgery image of a specific area of a subject; and a warning module directly or indirectly connected to the computer; wherein, the computer receives the pre-surgery image and transfer the pre-surgery image to a surgical planning information through the surgical planning program; wherein, the image capturing module continuously and instantly captures a surgery operation image of the specific area of the subject during the surgery of the specific area of the subject, and transfers the surgery operation image to the computer for the surgical planning program to compare the surgery operation image with the surgical planning information; wherein, under at least one of a first condition, a second condition and a third condition, the image capturing module captures a post-surgery image of a surgery area of the subject and transfers the post-surgery image to the computer, and the surgical planning program is updated according to the difference between the post-surgery image and the surgical planning information; wherein, under the first condition, the surgery operation image matches the surgical planning information, the warning module generates a first warning signal to a surgery operator; the surgery operator continues the surgery to make the surgery operation image exceed the surgical planning information, and completes the surgery; wherein, under the second condition, the surgery operation image does not match and not exceed the surgical planning information, and a surgery operator intends to stop the surgery, the warning module generates a second warning signal to the surgery operator; the surgery operator confirms to stop the surgery and makes the surgery operation image not match the surgical planning information, and completes the surgery; wherein, under the third condition, the surgery operation image exceed the surgical planning information, the warning module generates a third warning signal to a surgery operator; and the surgery operator confirms to stop the surgery.

Preferably, the surgical planning system further comprises an evaluating module, the evaluating module directly or indirectly connected to the computer and comprising an evaluation platform, and the evaluation platform provides a data-to-be-evaluated for at least one examiner performing the evaluation; wherein the data-to-be-evaluated comprises the post-surgery image and the surgical planning information; if at least one examiner confirms the data-to-be-evaluated, the surgical planning program will be updated. Wherein, through the evaluating module, the updating to the surgical planning program has to be confirmed by at least one professional examiner, and therefore the updating to the surgical planning program is examined or approved properly.

Preferably, when the surgery is performed on the specific area of the subject, the surgical planning information is updated through the surgical planning program according to the surgery operation image. Whereby, the surgical information is instantly updated according to the real-time surgery operation image, so that the system can provide the surgery operator with instant and proper surgical planning information.

Preferably, the surgical planning system further comprises a dose detecting module directly or indirectly connected to the computer for detecting a surgery dose applied to the specific area; wherein the surgical planning information comprises the information of the planned dose; when the surgery dose does not match the planned dose, the warning module generates a warning signal to the surgery operator; when the surgery operator confirms or adjusts the surgery dose to an applying dose and makes the applying dose not matching the planned dose and continues the surgery, the dose detecting module will transfer the information of the applying dose to the computer, and the surgical planning program will be updated according to the difference between the applying dose and the planned dose. Wherein, through the dose detecting module, the applying dose applied during the surgery is monitored. Furthermore, by updating the surgical planning system, a more suitable planned dose can be estimated.

In another broad aspect, there is provided a surgical planning method comprising: capturing a pre-surgery image of a specific area of a subject; transferring the pre-surgery image to a computer, wherein the computer comprises an accessible non-transitory memory, the accessible non-transitory memory comprises a surgical planning program, and the pre-surgery image is transformed to a surgical planning information through the surgical planning program; instantly capturing a surgery operation image of the specific area of the subject, transferring the surgery operation image to the computer, and comparing the surgery operation image with the surgical planning information through the surgical planning program; and under at least one of a first condition, a second condition and an optional third condition, capturing a post-surgery image and transferring the post-surgery image to the computer, and updating the surgical planning program according to the difference between the post-surgery image and the surgical planning information; wherein, under the first condition, the surgery operation image matches the surgical planning information, the warning module generates a first warning signal to a surgery operator; the surgery operator continues the surgery to make the surgery operation image exceed the surgical planning information, and completes the surgery; wherein, under the second condition, the surgery operation image does not match and not exceed the surgical planning information, and a surgery operator intends to stop the surgery, the warning module generates a second warning signal to the surgery operator; the surgery operator confirms to stop the surgery and makes the surgery operation image not match the surgical planning information, and completes the surgery; wherein, under the third condition, the surgery operation image exceeds the surgical planning information, the warning module generates a third warning signal to a surgery operator; the surgery operator confirms to stop the surgery.

In further another broad aspect, there is provided a non-transient computer-accessible medium having a surgical planning program stored therein, wherein the surgical planning program is provided to a computer through the non-transient computer-accessible medium, through applying the surgical planning program in the computer, the computer is configured to: receiving a pre-surgery image of a specific area of a subject and transforming the pre-surgery image to a surgical planning information; receiving a surgery operation image of the specific area and comparing the surgery operation image with the surgical planning information; and under at least one of a first condition, a second condition and an optional third condition, receiving a post-surgery image, and updating the surgical planning program according to the difference between the post-surgery image and the surgical planning information; wherein, under the first condition, the surgery operation image matches the surgical planning information, a warning module is provided to directly or indirectly connected to the computer, the warning module generates a first warning signal to a surgery operator; the surgery operator continues the surgery to make the surgery operation image exceed the surgical planning information, and completes the surgery; wherein, under the second condition, the surgery operation image does not match and not exceed the surgical planning information, and a surgery operator intends to stop the surgery, a warning module is provided to directly or indirectly connected to the computer, the warning module generates a second warning signal to the surgery operator; the surgery operator confirms to stop the surgery and makes the surgery operation image not match the surgical planning information, and completes the surgery; wherein, under the third condition, the surgery operation image exceeds the surgical planning information, a warning module is provided to directly or indirectly connected to the computer, the warning module generates a third warning signal to a surgery operator; the surgery operator confirms to stop the surgery.

Other advantages of the present invention will become apparent from the following descriptions taken in conjunction with the accompanying drawings wherein certain embodiments of the present invention are set forth by the way of illustration and examples.

### BRIEF DESCRIPTION OF THE DRAWINGS

The foregoing aspects and many of the accompanying advantages of this invention will become more readily appreciated as the same becomes better understood by reference to the following detailed descriptions, when taken in conjunction with the accompanying drawings, wherein:

FIG. 1 is schematic drawing showing the structure of the surgical planning system according to one of preferred embodiments of the present invention; and

FIG. 2 is schematic drawing showing the process flow of the surgical planning method according to one of preferred embodiments of the present invention.

### DESCRIPTION OF THE PREFERRED EMBODIMENT

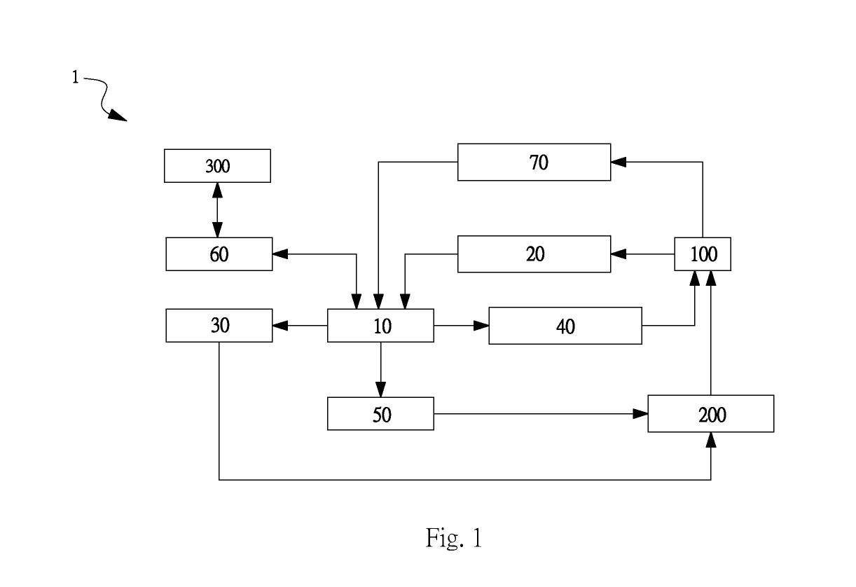

Please refer to FIG. 1 which shows the structure/framework of the surgical planning system 1 according to one of preferred embodiments of the present invention. The surgical planning system 1 comprises a computer 10, an image capturing module 20 and a warning module 30. The computer 10 comprises an accessible non-transitory memory and a surgical planning program, and the surgical planning program is stored in the accessible non-transitory memory. The image capturing module 20 is directly or indirectly connected to the computer 10, and comprises at least one of a camera and a video camera to capture a pre-surgery image of a specific area of a subject 100. Wherein, the subject 100 is a patient under surgery treatment. For example, when the surgical planning system 1 of the present invention is applied to the ophthalmology surgery, the subject 100 will be a surgery receiver, the specific area will be the eyes of the surgery receiver, and the pre-surgery image will be the eye image of the surgery receiver. In another example, when the surgical planning system 1 of the present invention is applied to the dermatological treatment, specifically to the feature of the facial skin, such as facial skin ulcer, acne, scar, wound or other features, the subject 100 will be a treatment receiver, the specific area will be partial or all of the facial skin of the treatment receiver, and the pre-surgery image will be the facial skin image of the treatment receiver. Preferably, in an optional example, the camera or the video camera is capable of emitting infrared ray or x-ray. Preferably, the image capturing module 20 has an eye topography apparatus and therefore makes the surgical planning system 1 of the present invention can be specifically used for ophthalmology surgery. Wherein, the background information of the eye topography apparatus is disclosed in the TW Patent Publication No. 201008544 (described as Topolyzer), and will no longer be repeated here. Further, in other examples, the image capturing module 20 optionally comprises a wavefront aberration measuring apparatus, an ultrasound scan device, a tomoscan apparatus, or an Endoscopy. It should be understood that the surgical planning system 1 of the present invention is not to be limited to the particular surgery or treatment disclosed above.

The computer 10 receives the pre-surgery image and transforms the pre-surgery image to a surgical planning information through the surgical planning program. Preferably, the surgical planning information comprises a planned surgery position and size corresponding to the specific area, and optionally comprises a planned dose, an estimated surgery time, a surgery operating step, or the information of a surgery comment. Preferably, the surgical planning information comprises the pre-surgery image. When the specific area of the subject 100 is performed the surgery, the image capturing module 20 will continuously and instantly capture a surgery operation image of the specific area of the subject 100. In this respect, the surgery operation image is captured in real-time corresponding to the real-time condition of the surgery. Further, the image capturing module 20 transfers the surgery operation image to the computer 10, and the surgery operation image is compared with the surgical planning information through the surgical planning program. More specifically, the surgery operation image is updated corresponding to the newest image of the subject 100. In one preferred example of the present invention, when the surgery is performed on the specific area of the subject 100, the surgical planning information can be updated through the surgical planning program according to the surgery operation image. With the instant update of the surgical planning information, influences from the surface feature and other surface environmental factors of the specific area of the subject 100 can be reduced, and the surgery operator 200 can also obtain instant and suitable surgical planning information. Optionally, the surgical planning system 1 further comprises a surgery applying equipment 40. The surgery applying equipment 40 is directly or indirectly connected to the computer 10 and is capable of automatically applying the surgery to the subject 100 according to the surgical planning information. Optionally, the surgical planning system 1 further comprises a display device 50. The display device 50 is directly or indirectly connected to the computer 10 for displaying the at least one of the surgical planning information and the surgery operation image, and the surgery operator 200 can handle the surgical detail through the display device 50.

The warning module 30 is directly or indirectly connected to the computer 10, and comprises at least one of a vocal device, a luminous device and a vibrational device. Wherein, the vocal device is used for generating a sound wave signal; the luminous device is used for generating an optical wave signal; and the vibrational device (such as a vibration motor) is used for generating a vibration wave signal. When the surgery operation image matches the surgical planning information, the warning module 30 will generate a first warning signal to the surgery operator 200. Then, the surgery operator 200 can make a decision of finishing or continuing the surgery. Under a first condition, if the surgery operator 200 continues the surgery, makes the surgery operation image exceed the surgical planning information, and completes the surgery, the image capturing module 20 will capture a post-surgery image of a surgery area of the subject 100 and transfer the post-surgery image to the computer 10. The surgical planning program is updated according to the difference between the post-surgery image and the surgical planning information. Under a second condition, when the surgery operation image does not match and not exceed the surgical planning information, and the surgery operator 200 intends to stop the surgery, the warning module 30 will generate a second warning signal to the surgery operator 200; when the surgery operator 200 confirms to stop the surgery to make the surgery operation image does not match the surgical planning information, and completes the surgery, the image capturing module 20 will capture a post-surgery image of a surgery area of the subject 100 and transfer the post-surgery image to the computer 10. The surgical planning program is updated according to the difference between the post-surgery image and the surgical planning information. Under an optional third condition, when the surgery operation image exceeds the surgical planning information, the warning module 30 will generate a third warning signal to the surgery operator 200; and no matter the surgery operator 200 continues the surgery or not, the surgery operation image is no longer matching the surgical planning information. When the surgery operator 200 confirms to stop the surgery, the image capturing module 20 will capture a post-surgery image of a surgery area of the subject 100 and transfer the post-surgery image to the computer 10. The surgical planning program is updated according to the difference between the post-surgery image and the surgical planning information. Wherein, the first warning signal, the second warning signal and the third warning signal comprises at least one of a sound wave, an optical wave and a vibration wave. In one example, the first warning signal, the second warning signal and the third warning signal are different. For instance, when the first warning signal, the second warning signal and the third warning signal are all optical wave signals, the first warning signal is a green light, the second warning signal is a blue light and the third warning signal is a red light. In a preferred example, under at least one of the first condition, the second condition and the third condition, the surgery operator 200 can input a surgery comment, which will be stored to the accessible non-transitory memory of the computer 10, for recording relevant considerations of the surgery operator 200 of the surgery operation. The surgical planning program will include the information of the surgery comment when updating, and the surgical planning system 1 of the present invention will provide the surgery comment to the later surgery operator 200 in the future related surgery.

For further thoroughly explaining, “when the surgery operation image does not match and not exceed the surgical planning information” mentioned above in the second condition means that: under the same comparison standard and condition, if the surgical planning information defines a surgical planning area which can be transformed into position coordinate information, the position coordinate of at least one point in the surgery operation image does not match the position coordinate of at least one point in the surgical planning area, and the position coordinate of any point in the surgery operation image does not exceed the boundary of the surgical planning area. Similarly, “when the surgery operation image exceeds the surgical planning information” mentioned above in the third condition means that: the position coordinate of at least one point in the surgery operation image exceeds the boundary of the surgical planning area. Wherein, it should be noted that the difference between the surgery operation image and the surgical planning information should have a default tolerance range. For example, in a precision surgery, a default tolerance range is 0.001 mm, and only when the difference between the surgery operation image and the surgical planning information exceeds the default tolerance range, the surgical planning program will perform the update according to the difference. However, the default tolerance range is not limited to said scale definition.

Preferably, the surgical planning system 1 can further optionally comprises an evaluating module 60. The evaluating module 60 is directly or indirectly connected to the computer 10, and comprises an evaluation platform. The evaluation platform provides a data-to-be-evaluated for at least one examiner 300 performing the evaluation; wherein the data-to-be-evaluated comprises the post-surgery image and the surgical planning information stored in the accessible non-transitory memory. Under at least one of the first condition, the second condition and the third condition, and before updating the surgical planning program, the evaluating module 60 can read the data-to-be-evaluated from the accessible non-transitory memory and transfer the data-to-be-evaluated to the evaluation platform for displaying to an examiner 300. When the examiner 300 confirms the data-to-be-evaluated through the evaluation platform, the surgical planning program will be updated. Wherein, if there are more than one examiner 300, the evaluating module 60 can set an updating approval ratio. When the averaged approval ratio from multiple examiners 300 is not less than the updating approval ratio, the surgical planning program will be updated in accordance with the data-to-be-evaluated. Wherein, he averaged approval ratio defined herein is the ratio of the amount of the examiner approving the evaluating data and the amount of total examiner. The updating approval ratio is a value from 0 to 1. Preferably, the updating approval ratio is not less than 0.5. Preferably, the data-to-be-evaluated can further optionally comprises the surgery comment, and the examiner 300 can confirm, modify or delete the surgery comment to be an evaluated comment. Preferably, the evaluated comment may comprise an extra evaluating comment denoted by the examiner 30 and recording the relevant considerations of the examiner 30. Preferably, the surgical planning information optionally comprises at least one evaluating comment, and the surgical planning system 1 of the present invention will provide the evaluating comment to the later surgery operator 200 in the future related surgery.

Preferably, the surgical planning system 1 optionally comprises a dose detecting module 70. The dose detecting module 70 is directly or indirectly connected to the computer 10 for detecting a surgery dose applied to the specific area. Wherein, the surgical planning information comprises the information of the planned dose, and when the surgery dose does not match the planned dose, the warning module 30 will generate a warning signal to the surgery operator 200. When the warning module 30 generates a warning signal, and the surgery operator 200 confirms or adjusts the surgery dose to an applying dose, makes the applying dose not matching the planned dose, and continues the surgery, the dose detecting module 70 will transfer the information of the applying dose to the computer 10, and the surgical planning program will be updated according to the difference between the applying dose and the planned dose. In a preferred example, the evaluating data optionally comprises the applying dose and the planned dose. It should be noted that the word “dose” of the surgery dose, the planned dose and the applying dose includes the “medical dose” or “energy dose.” The “medical dose” comprises the density, amount or dosing time of the medicine. The “energy dose” comprises the strength, frequency or dosing time of the energy; wherein the sort of the “energy dose” comprises, but does not be limited to, laser energy, optical energy, or wave/vibration energy.

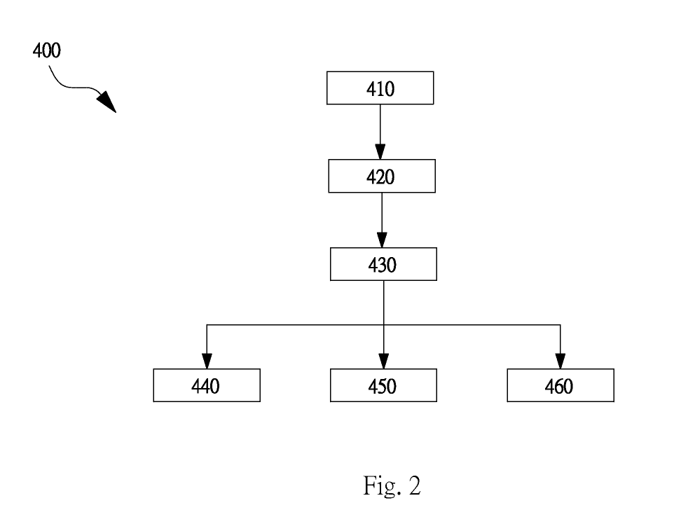

As shown in FIG. 2 (please also refer to FIG. 1), according to the surgical planning system 1 described above, the surgical planning method 400 of the present invention comprises the steps of:

Step 410: capturing a pre-surgery image of a specific area of a subject 100 through an image capturing module 20;

Step 420: transferring the pre-surgery image to a computer 10, wherein, the computer 10 comprises an accessible non-transitory memory, the accessible non-transitory memory comprises a surgical planning program, and the pre-surgery image is transformed to a surgical planning information through the surgical planning program;

Step 430: instantly capturing a surgery operation image of the specific area of the subject 100 through the image capturing module 20, transferring the surgery operation image to the computer 10, and comparing the surgery operation image with the surgical planning information through the surgical planning program;

Step 440: when the surgery operation image matches the surgical planning information, the warning module 30 will generate a first warning signal to a surgery operator 200; if the surgery operator 200 continues the surgery to make the surgery operation image exceed the surgical planning information, and completes the surgery, the image capturing module 20 will capture a post-surgery image and transferring the post-surgery image to the computer 10, and the surgical planning program will be updated according to the difference between the post-surgery image and the surgical planning information;

Step 450: when the surgery operation image does not match and not exceed the surgical planning information, and the surgery operator 200 intends to stop the surgery, the warning module 30 will generate a second warning signal to the surgery operator 20; if the surgery operator 200 confirms to stop the surgery and makes the surgery operation image not match the surgical planning information, and completes the surgery, the image capturing module 20 will capture a post-surgery image and transferring the post-surgery image to the computer 10, and the surgical planning program will be updated according to the difference between the post-surgery image and the surgical planning information;

Optional step 460: when the surgery operation image exceeds the surgical planning information, the warning module 30 will generate a third warning signal to the surgery operator 200; and no matter the surgery operator 200 continues the surgery or not, the surgery operation image is no longer matching the surgical planning information. When the surgery operator 200 confirms completing the surgery, the image capturing module 20 will capture a post-surgery image and transferring the post-surgery image to the computer 10, and the surgical planning program will be updated according to the difference between the post-surgery image and the surgical planning information.

It should be noted that, in addition to the technical features mentioned above, the surgical planning method 400 can also optionally comprises all or partial features of the surgical planning system 1. Preferably, the surgical planning method 400 optionally comprises at least one of the following features:

1. When the surgery is performed on the specific area of the subject 100, the surgical planning information is updated according to the surgery operation image through the surgical planning program.

2. Before updating the surgical planning program, a data-to-be-evaluated has to be displayed through an evaluation platform of an evaluating module 60, and the surgical planning program will be updated only after the data-to-be-evaluated is evaluated and confirmed by an examiner 300; the data-to-be-evaluated comprises the post-surgery image and the pre-surgery surgical planning information.

3. Detecting a surgery dose applied to the specific area through a dose detecting module 70; wherein, the surgical planning information comprises information of the planned dose. When the surgery dose does not match the planned dose, the warning module 30 will generate a warning signal. When the warning module 30 generates a warning signal and the surgery operator 200 confirms or adjusts the surgery dose to a applying dose and makes the applying dose not matching the planned dose, and executes the surgery, the dose detecting module 70 will transfer the information of the applying dose to the computer 10 and the surgical planning program will be updated according to the difference between the applying dose and the planned dose.

4. Automatically operating a surgery applying equipment 40 to perform the surgery to the subject 100 according to the surgical planning information, so that the post-surgery image matches to the surgical planning information

It should be noted that “directly or indirectly connect” described in the application means: two devices (such as the image capturing module 20 and the computer 10) are electrically connected directly or connected through network transmission technology indirectly.

As above-mentioned surgical planning system and method of the present invention, the present invention is adapted to generate the surgical planning information and warning signals. The surgical planning information is a reference or guidance of a surgery operation and hence achieves an assistant effect to the surgery operation. The warning signal is capable of warning the surgery operator 200, so that the surgery operator 200 is noticed to make judgments/diagnoses for specific surgery conditions, and hence improve surgery quality. The surgical planning system and method of the present invention can further update the surgical planning program according to the first condition, the second condition and the third condition. The surgical planning program can be updated according to the practicing experience and judgment of the surgery operator 200 and makes the updated surgical planning program generating a more suitable surgical planning information. Optionally, when a surgery operation is performed, the surgical planning information can be also updated according to a current surgery operation image. Therefore, the present invention has the effect of instantly updating the surgical information according to the real-time surgery operation image, and provides the surgery operator 200 with instant and suitable surgical planning information. Optionally, the surgical planning system and method comprise the dose detecting module 70. When the surgery dose does not match the planned dose, the dose detecting module 70 can generate a warning signal to the surgery operator 200, and hence has the effect of assisting and warning the surgery operator 200. When the surgery dose does not match the planned dose and the surgery is executed, the surgical planning program will be updated according to the difference between the applying dose and the planned dose, and hence the updated surgical planning program will have the effect of generating more suitable surgical planning information. Optionally, the surgical planning system and method comprise the evaluating module 60. Through the evaluating module 60, the updating to the surgical planning program has to be confirmed by at least one professional examiner 300, and therefore the updating to the surgical planning program is examined or approved properly.

More specifically, the surgical planning system and method according to the present invention execute the surgical planning program through the processor in the computer 10 to interact with the computer 10, the image capturing module 20, the warning module 30, the surgery applying equipment 40, the display device 50, the evaluating module 60 and the dose detecting module 70.

Acts of one or more of the systems and methods described herein may be embodied as the surgical planning program which is deemed as computer-readable instructions, stored in a non-transient computer-accessible medium, for example. Wherein, said surgical planning program are executed by a computer with a processor, so that the computer or processor is configured to perform the one or more of the foregoing methods herein

While the invention can be subject to various modifications and alternative forms, a specific example thereof has been shown in the drawings and is herein described in detail. It should be understood, however, that the invention is not to be limited to the particular form disclosed, but on the contrary, the invention is to cover all modifications, equivalents, and alternatives falling within the spirit and scope of the appended claims.

What is claimed is:

1. A surgical planning system, comprising:
a computer having an accessible non-transitory memory and a surgical planning program, the surgical planning program is stored in the accessible non-transitory memory;
an image capturing module directly or indirectly connected to the computer, and comprising at least one of a camera and a video camera to capture a pre-surgery image of a specific area of a subject; and
a warning module directly or indirectly connected the computer;
wherein, the computer receives the pre-surgery image and transfers the pre-surgery image to a surgical planning information through the surgical planning program;
wherein, the image capturing module continuously and instantly captures a surgery operation image of the specific area of the subject during the surgery of the specific area of the subject, and transfers the surgery operation image to the computer for the surgical planning program to compare the surgery operation image with the surgical planning information;
wherein, under at least one of a first condition, a second condition and a third condition, the image capturing module captures a post-surgery image of a surgery area of the subject and transfers the post-surgery image to the computer, and the surgical planning program is updated according to the difference between the post-surgery image and the surgical planning information;
wherein, under the first condition, the surgery operation image matches the surgical planning information, the warning module generates a first warning signal to a surgery operator; the surgery operator continues the surgery to make the surgery operation image exceed the surgical planning information, and completes the surgery;
wherein, under the second condition, the surgery operation image does not match and not exceed the surgical planning information, and a surgery operator intends to stop the surgery, the warning module generates a second warning signal to the surgery operator; the surgery operator confirms to stop the surgery and makes the surgery operation image not match the surgical planning information, and completes the surgery;
wherein, under the third condition, the surgery operation image exceeds the surgical planning information, the warning module generates a third warning signal to a surgery operator; and the surgery operator confirms to stop the surgery.

2. The surgical planning system according to claim 1, further comprising an evaluating module, the evaluating module directly or indirectly connected to the computer and comprising an evaluation platform, and the evaluation platform provides a data-to-be-evaluated for at least one examiner performing the evaluation; wherein the data-to-be-evaluated comprises the post-surgery image and the surgical planning information; if at least one examiner confirms the data-to-be-evaluated, the surgical planning program will be updated.

3. The surgical planning system according to claim 1, wherein when the surgery is performed on the specific area of the subject, the surgical planning information is updated through the surgical planning program according to the surgery operation image.

4. The surgical planning system according to claim 2, wherein when the surgery is performed on the specific area of the subject, the surgical planning information is updated through the surgical planning program according to the surgery operation image.

5. The surgical planning system according to claim 1, wherein the surgical planning information comprises a planned surgery area corresponding to the specific area, information of a simulated treatment position or size, and optionally comprises a planned dose, an estimated surgery time, a surgery operating step, or information of a surgery comment.

6. The surgical planning system according to claim 5, further comprising a dose detecting module directly or indirectly connected to the computer for detecting a surgery dose applied to the specific area; wherein the surgical planning information comprises the information of the planned dose; when the surgery dose does not match the planned dose, the warning module generates a warning signal to the surgery operator; when the surgery operator confirms or adjusts the surgery dose to an applying dose and makes the applying dose not matching the planned dose and continues the surgery, the dose detecting module will transfer the information of the applying dose to the computer, and the surgical planning program will be updated according to the difference between the applying dose and the planned dose.

7. The surgical planning system according to claim 1, further comprising a surgery operating equipment, the surgery operating equipment directly or indirectly connected to the computer and acting according to the surgical planning information.

8. The surgical planning system according to claim 1, wherein the image capturing module further comprises at least one of an eye topography apparatus, a wavefront aberration measuring apparatus, an ultrasound scan device, a tomoscan apparatus and an endoscopy.

9. The surgical planning system according to claim 1, further comprising a display device, the display device directly or indirectly connected to the computer for displaying at least one of the surgical planning information and the surgery operation image.

10. A surgical planning method, comprising:
capturing a pre-surgery image of a specific area of a subject;
transferring the pre-surgery image to a computer, wherein the computer comprises an accessible non-transitory memory, the accessible non-transitory memory comprises a surgical planning program, and the pre-surgery image is transformed to a surgical planning information through the surgical planning program;
instantly capturing a surgery operation image of the specific area of the subject, transferring the surgery operation image to the computer, and comparing the surgery operation image with the surgical planning information through the surgical planning program; and
under at least one of a first condition, a second condition and an optional third condition, capturing a post-surgery image and transferring the post-surgery image to the computer, and updating the surgical planning program according to the difference between the post-surgery image and the surgical planning information;
wherein, under the first condition, the surgery operation image matches the surgical planning information, the warning module generates a first warning signal to a surgery operator; the surgery operator continues the surgery to make the surgery operation image exceed the surgical planning information, and completes the surgery;
wherein, under the second condition, the surgery operation image does not match and not exceed the surgical planning information, and a surgery operator intends to stop the surgery, the warning module generates a second warning signal to the surgery operator; the surgery operator confirms to stop the surgery and makes the surgery operation image not match the surgical planning information, and completes the surgery;
wherein, under the third condition, the surgery operation image exceeds the surgical planning information, the warning module generates a third warning signal to a surgery operator; the surgery operator confirms to stop the surgery.

11. The surgical planning method according to claim 10, wherein an evaluating module is provided, the evaluating module is directly or indirectly connected to the computer and comprises an evaluation platform; before updating the surgical planning program, a data-to-be-evaluated is presented on the evaluation platform for at least one examiner performing the evaluation; when the at least one examiner confirms the data-to-be-evaluated, applying the updating of the surgical planning program.

12. The surgical planning method according to claim 10, wherein when the surgery is performed on the specific area of the subject, the surgical planning information is updated according to the surgery operation image through the surgical planning program.

13. The surgical planning method according to claim 11, wherein when the surgery is performed on the specific area of the subject, the surgical planning information is updated according to the surgery operation image through the surgical planning program.

14. The surgical planning method according to claim 10, wherein the surgical planning information comprises a planned surgery area corresponding to the specific area, information of a simulated treatment position or size, and optionally comprises a planned dose, an estimated surgery time, a surgery operating step, or information of a surgery comment.

15. The surgical planning method according to claim 14, wherein the surgical planning information comprises information of the planned dose; a dose detecting module is provided for detecting a surgery dose applied to the specific area; when the surgery dose does not match the planned dose, the warning module will generate a warning signal; after a surgery operator confirms or adjusts the surgery dose to a applying dose and makes the applying dose not matching the planned dose, and executes the surgery, the dose detecting module will transfer the information of the applying dose to the computer, and the surgical planning program will be updated according to the difference between the applying dose and the planned dose.

16. The surgical planning method according to claim 10, wherein the image capturing module further comprises at least one of an eye topography apparatus, a wavefront aberration measuring apparatus, an ultrasound scan device, a tomoscan apparatus and an endoscopy.

17. The surgical planning method according to claim 10, a display device is provided, the display device directly or indirectly connected to the computer for displaying at least one of the surgical planning information and the surgery operation image.

18. The surgical planning method according to claim 10, wherein a surgery operating equipment is provided, the surgery operating equipment directly or indirectly connected to the computer and acting according to the surgical planning information.

19. A non-transient computer-accessible medium having a surgical planning program stored therein, wherein the surgical planning program is provided to a computer through the non-transient computer-accessible medium, through applying the surgical planning program in the computer, the computer is configured to:
receiving a pre-surgery image of a specific area of a subject and transforming the pre-surgery image to a surgical planning information;
receiving a surgery operation image of the specific area and comparing the surgery operation image with the surgical planning information; and
under at least one of a first condition, a second condition and an optional third condition, receiving a post-surgery image, and updating the surgical planning program according to the difference between the post-surgery image and the surgical planning information;
wherein, under the first condition, the surgery operation image matches the surgical planning information, a warning module is provided to directly or indirectly connected to the computer, the warning module generates a first warning signal to a surgery operator; the surgery operator continues the surgery to make the surgery operation image exceed the surgical planning information, and completes the surgery;
wherein, under the second condition, the surgery operation image does not match and not exceed the surgical planning information, and a surgery operator intends to stop the surgery, a warning module is provided to directly or indirectly connected to the computer, the warning module generates a second warning signal to the surgery operator; the surgery operator confirms to stop the surgery and makes the surgery operation image not match the surgical planning information, and completes the surgery;
wherein, under the third condition, the surgery operation image exceeds the surgical planning information, a warning module is provided to directly or indirectly connected to the computer, the warning module generates a third warning signal to a surgery operator; the surgery operator confirms to stop the surgery.
